(19) United States
(12) Patent Application Publication
(10) Pub. No.: US 2025/0134741 A1
(43) Pub. Date: May 1, 2025

(54) ERGONOMIC SURGICAL INTERVENTION TABLES

(71) Applicant: Mayo Foundation for Medical Education and Research, Rochester, MN (US)

(72) Inventors: Mandeep Singh, LaCrosse, WI (US); Samuel J. Asirvatham, Rochester, MN (US)

(21) Appl. No.: 18/837,102

(22) PCT Filed: Feb. 7, 2023
(86) PCT No.: PCT/US2023/012499

§ 371 Date: Aug. 8, 2024

Related U.S. Application Data
(60) Provisional application No. 63/307,846, filed on Feb. 8, 2022.

(51) Int. Cl. A61G 13/08; A61B 46/00

(52) U.S. Cl. CPC A61G 13/08; A61B 46/00

(57) ABSTRACT

This document describes surgical instrument tables. For example, this document describes ergonomic surgical intervention tables that are user-friendly and easy-to-deploy. In some embodiments, the surgical intervention table is designed as an attachment for an existing catheterization or operating table. The mounting of the table is flexible as the surgical intervention table can be mounted either at the foot side or lateral side of the table, away from the operator, and can be folded back underneath it with push of a button.

### CROSS-REFERENCE TO RELATED APPLICATIONS

This application claims the benefit of U.S. Provisional Application Ser. No. 63/307,846 filed Feb. 8, 2022. The disclosure of the prior application is considered part of (and is incorporated by reference in) the disclosure of this application.

### BACKGROUND

### 1. Technical Field

This document relates to surgical instrument tables. For example, this document relates to ergonomic surgical intervention tables that are user-friendly and easy-to-deploy. In some embodiments, the surgical intervention table is designed as an attachment for an existing catheterization or operating table. The mounting of the table is flexible as the surgical intervention table can be mounted either at the foot side or lateral side of the table, away from the operator, and can be folded back underneath it with push of a button.

### 2. Background Information

During diagnostic and interventional coronary, electrophysiology, peripheral vascular procedures and all types of surgeries, there is currently a freestanding table that is placed behind the operator that carries all the catheters, wires, stents, or surgical equipment. This is ergonomically inefficient and adds to occupational hazard of the operators as they pivot back and forth hundreds of times during the procedure to gather the equipment and move them to the operating or catheter lab table located in front of the operator. During a routine diagnostic coronary angiography procedure, operators pivot on an average 25 times, for percutaneous coronary interventions more than 50 times and for transcatheter aortic valve replacement (TVAR) more than 100 times, underscoring the importance of having the table in front of the operator during such long and complex procedures.

In 2007, approximately 1.1 million cardiac catheterization and 622,000 percutaneous coronary interventions (PCI) were performed in the United States. More than 40,000 transcutaneous aortic valve replacements have been performed worldwide and the numbers are far greater when one includes other structural interventions involving other valves, pediatric, peripheral vascular interventions, surgeries, ablations, and device implantations performed by electrophysiologists. With the aging population and expansion to include highly complex and long procedures, there is an urgent need to develop simple and effective interventions to lower the health risk of employees engaged in such procedures as these numbers will continue to grow.

### SUMMARY

This document describes surgical instrument tables. For example, this document describes ergonomic surgical intervention tables that are user-friendly and easy-to-deploy. In some embodiments, the surgical intervention table is designed as an attachment for an existing catheterization or operating table. The surgical intervention table can be mounted at the foot side of the table and can be folded back underneath it with push of a button.

Particular embodiments of the subject matter described in this document can be implemented to realize one or more of the following advantages. First, there are currently no surgical instrument table designs that fit the description of the surgical instrument tables described herein. There is an unmet need for such tables as they provide an ergonomically efficient way to deliver medical care to the staff working in the departments of cardiology, radiology, and surgery.

In some embodiments, the surgical instrument tables described herein mirror the dimensions of the current freestanding tables. The surgical instrument tables will use the unused or dead space from below the groin of the patient to the end of the catheter lab table without using extra space in catheter or EP labs. Currently, only bulky freestanding tables are available that can be moved around in the operating rooms or catheterization laboratories but occupy additional space in already crowded procedure rooms.

A study recently performed by the inventors demonstrated the magnitude of musculoskeletal problems among employees working in these high-risk areas of radiology, catheter and echo labs. In this multisite, case-control study that involved physicians and non-physician allied health staff, 55% of personnel working in radiology and cardiology departments reported musculoskeletal pain, 30% sought medical care for pain, and 29% had pain at the time of the study. Despite being younger and having worked for fewer years, technicians and nurses reported a higher prevalence of work-related musculoskeletal pain. The high prevalence of musculoskeletal pain presents an opportunity to improve the work-related health of employees and the surgical instrument tables described herein offer solutions to that problem for employees working in high-risk surgical and interventional environment.

Additional advantages of the surgical instrument tables described herein are the compact design and the ease of use of the design.

In one aspect, this disclosure is directed to a surgical instrument table that includes a base; a casing rotatably coupled to the base; a first table section; a support mechanism coupling the first table section to the casing; and a second table section slidably coupled to the first table section.

Such a surgical instrument table may optionally include one or more of the following features. The first and second table sections may be slidable into and out of an interior space of the casing. The casing may be positionable adjacent to an underside surface of the base. The first and second table sections may be pivotable in relation to the casing. The first and second table sections may be rotatable 180° relative to the base. One or more of the movements of the casing and the first and second table sections may be motorized. One or more of the movements of the casing and the first and second table sections may be manually actuated.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more exemplary embodiments of the invention are set forth in the accompanying drawings and the description herein. Other features, objects, and advantages of exemplary devices described herein will be apparent from the description and drawings, and from the claims.

### BRIEF DESCRIPTION OF THE DRAWINGS

The present description will be further explained with reference to the appended Figures, wherein like structure is referred to by like numerals throughout the several views, and wherein:

FIG. 1 shows a typical operating room or catheterization lab arrangement. An example ergonomic surgical intervention table in accordance with embodiments disclosed herein is also shown.

FIG. 2 shows a reach zone diagram.

FIG. 3 shows the reach zone diagram superimposed on the operating room or catheterization lab arrangement of FIG. 1.

FIG. 4 shows a perspective view of an example ergonomic surgical intervention table and an exploded view of the example ergonomic surgical intervention table.

FIGS. 5-9 are a sequence of illustrations that depict the configurations involved in setting up the example ergonomic surgical intervention table.

FIGS. 10-13 are a sequence of illustrations that depict the configurations involved in taking down (and storing) the example ergonomic surgical intervention table.

FIG. 14 depicts some additional accessories that can be used with the ergonomic surgical intervention table as described herein.

While the above-identified figures set forth various embodiments of the described subject matter, other embodiments are also contemplated. In all cases, this document presents the described subject matter by way of representation and not limitation. It should be understood that numerous other modifications and embodiments can be devised by those skilled in the art which fall within the scope and spirit of the present description.

### DETAILED DESCRIPTION OF EXAMPLE EMBODIMENTS

This document describes surgical instrument tables. For example, this document describes ergonomic surgical intervention tables (“ESIT”) that are user-friendly and easy-to-deploy. In some embodiments, the ESIT is designed as an attachment for an existing catheterization or operating table. The ESIT can be mounted at the foot side or on the side opposite to the operator and can be folded back underneath it with push of a button.

The ESIT can be positioned above a patient that is on an operating table. The instrument table of the ESIT is housed in a cassette that will hang by the side of the operating table and will not occupy any space in the catheterization laboratories and operating rooms. The height and the length of the instrument table are adjustable and mirror the dimensions of the current free-standing tables being used. The ESIT can swivel, and can be moved away from the patient in case of an emergency. The ESIT has rolled-up edges to prevent the equipment from falling from the table. The ESIT can be attached on either the left or right side, on the front or back of the existing catheterization or operating room table, to give flexibility to the operating personnel.

The ESIT will be built for either manual adjustment or provided with motorized option. With the motorized option, the equipment can be set in place automatically and rolled back to the original position once the procedure is complete. In some embodiments, the ESIT designs will include lightweight composite material to reduce the weight of the table.

The ESIT is an ergonomically efficient attachment for catheterization laboratory or for tables used in the operating rooms, which ensures comfort for lab professionals by taking in consideration the human-centric factors in addition to utility. Cath-lab professionals wear heavy personal radiation protective gear in the form of lead apron while performing procedures lengthening to several hours. The unfriendly design and placement of presently used instrument tables makes it harder for them to approach the equipment needed for the procedure. From workplace ergonomics point of view, the motions involved are not only relatively complex but are also repeated over several times during a session. These motions involve use of back muscles and tissues in a unilateral direction, which can alter their primary function in long term. Considering the resistance offered to motion by protective equipment, exposure to such physical stress is linked to neck, spine, hip, shoulder and knee injuries that are shortening careers for medical professionals and limiting their activities outside of work. ESIT targets to remove this major hindrance to an injury free career for professionals who work in the catheterization laboratories and operating rooms.

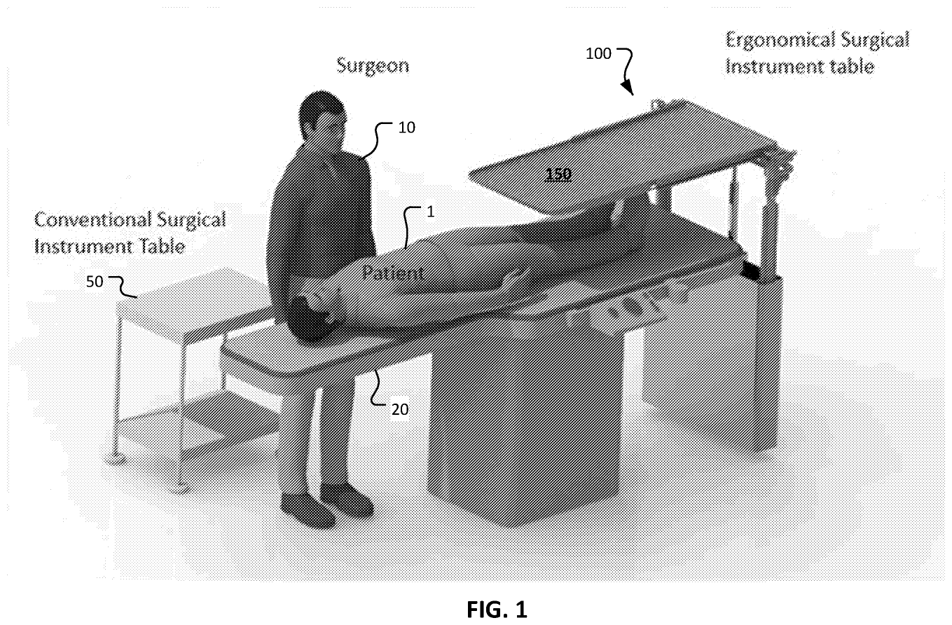

FIG. 1 depicts an example catheterization lab or operating room arrangement. The operator 10 (which represents any type of healthcare worker) typically stands along an operating table 20 on which a patient 1 is lying, and faces the patient 1.

The depicted arrangement also includes a conventional surgical instrument table 50. The conventional surgical instrument table 50 is typically positioned at least partially behind the operator 10 as shown. Accordingly, the operator 10 must pivot back and forth many times during the procedure to gather the instruments and equipment from the conventional surgical instrument table 50 and then to use them to work on the patient 1 lying on the operating or catheter lab table 20. This is obviously not a user-friendly, ergonomic arrangement because of the relative arrangement of the conventional surgical instrument table 50 in relation to the operator 10.

An ESIT (ergonomic surgical instrument table) 100 in accordance with some embodiments is also pictured. The ESIT 100 includes a table surface 150 on which surgical instruments are accessible for use by the operator 10. Advantageously, the table surface 150 is readily accessible by the operator 10. Accordingly, the use of the ESIT 100 can improve the ergonomics and efficiency of the operator 10 during a catheter or surgical procedure.

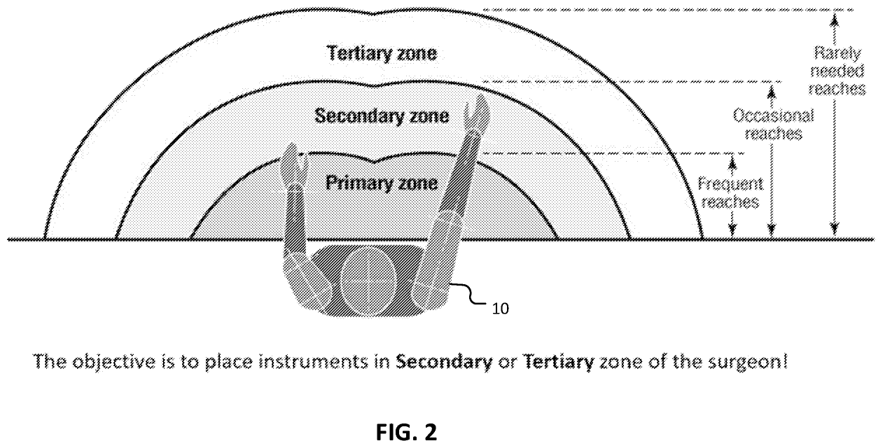

FIG. 2 shows a top view of the operator 10 and reach zones of the operator 10. Reach zones are the locations where work is done and are divided into a primary zone, a secondary zone, and a tertiary zone. From an ergonomic and efficiency standpoint, recurrent tasks and frequently used tools should be positioned within the primary zone. The secondary zone is where materials and tools used less frequently are placed. Everything can be reached within the secondary zone envelope by an outstretched arm. The tertiary zone requires additional bodily movement. Generally, infrequently used items can be stored in the tertiary zone.

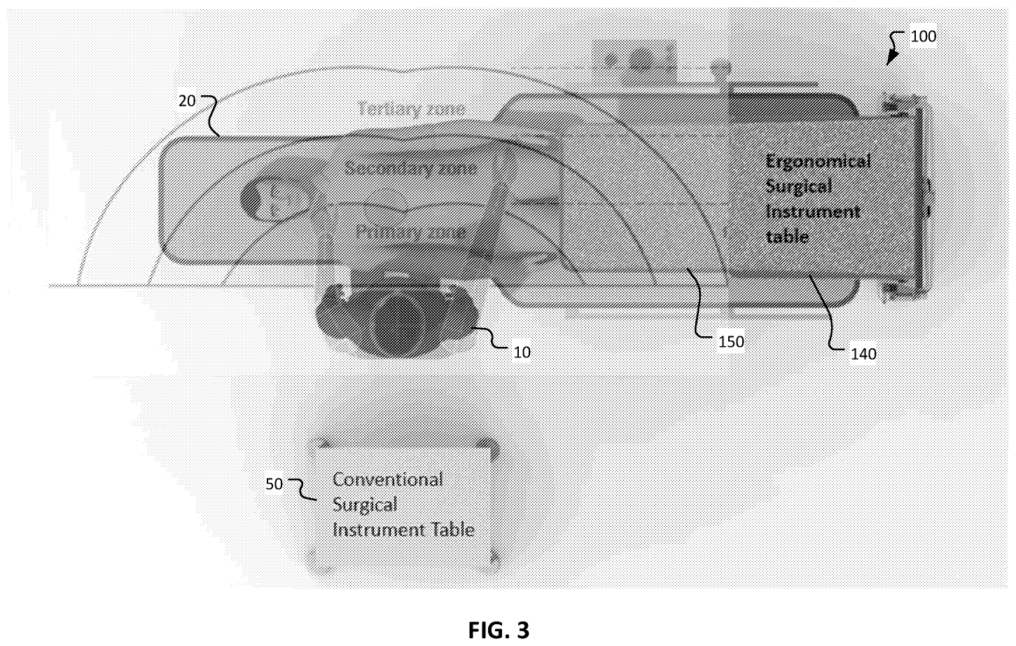

FIG. 3 shows the operator 10, the reach zones of the operator 10 (as per FIG. 2), the catheter lab table 20, the patient 1, the conventional surgical instrument table 50, and the ESIT 100. It can be seen that the table surface 150 of the ESIT 100 is within the secondary zone and the tertiary zone of the operator 10. In contrast, the conventional surgical instrument table 50 is way out of the reach zones of the operator 10. Accordingly, from ergonomic and efficiency standpoints, the ESIT 100 provides a much improved instrument table as compared to the conventional surgical instrument table 50.

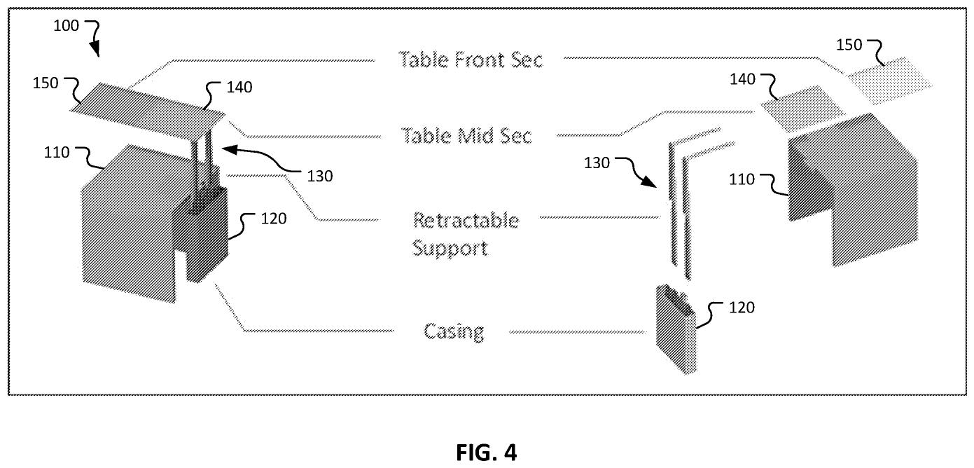

FIG. 4 shows the component parts of an example embodiment of the ESIT 100. The example ESIT 100 includes a base 110, a casing 120, a retractable support 130, a middle table section 140, and a front table section 150. The casing 120 contains the retractable support 130, the middle table section 140, and the front table section 150. The casing 120 is pivotably coupled to the base 110. The retractable support 130 is movably coupled to the casing 120. The middle table section 140 is pivotably and rotatably coupled to the retractable support 130. The front table section 150 slidably coupled to the middle table section 140.

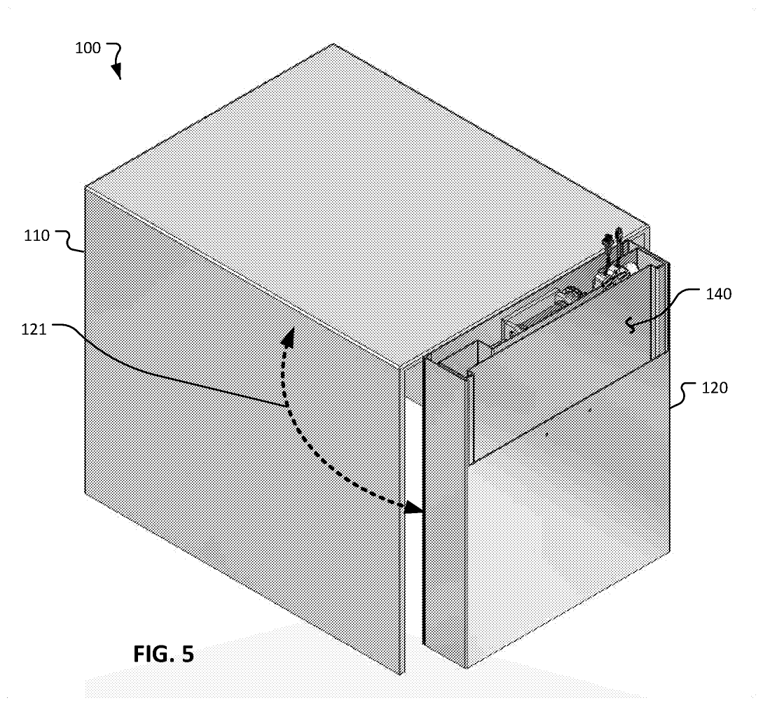

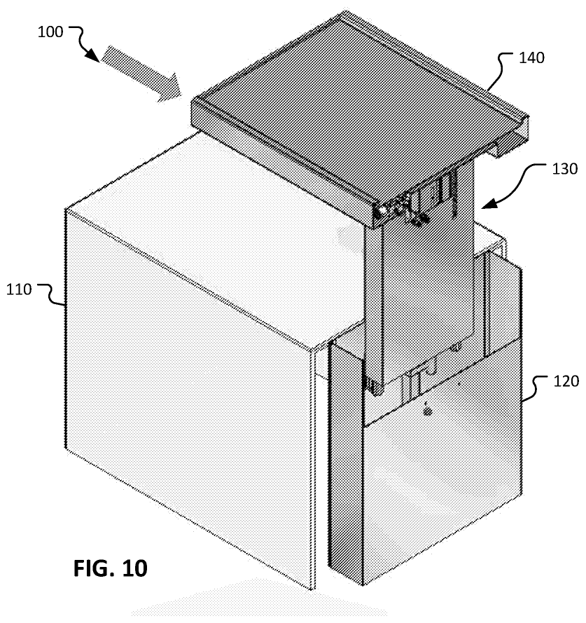

FIGS. 5-9 depict the sequential configurations involved in setting up the ESIT 100, and FIGS. 10-13 depict the sequential configurations involved in taking down (or storing) the ESIT 100. The motions involved in the sequential configurations can be motorized, or can be performed manually.

FIG. 5 shows that the casing 120 can be pivoted downward from the underside top of the base 110. When the ESIT 100 is in its storage configuration, the casing 120 is adjacent to the underside top of the base 110. The arrow 121 depicts the 90° pivoting of the casing 120 relative to the base 110 from its storage configuration to its operable configuration.

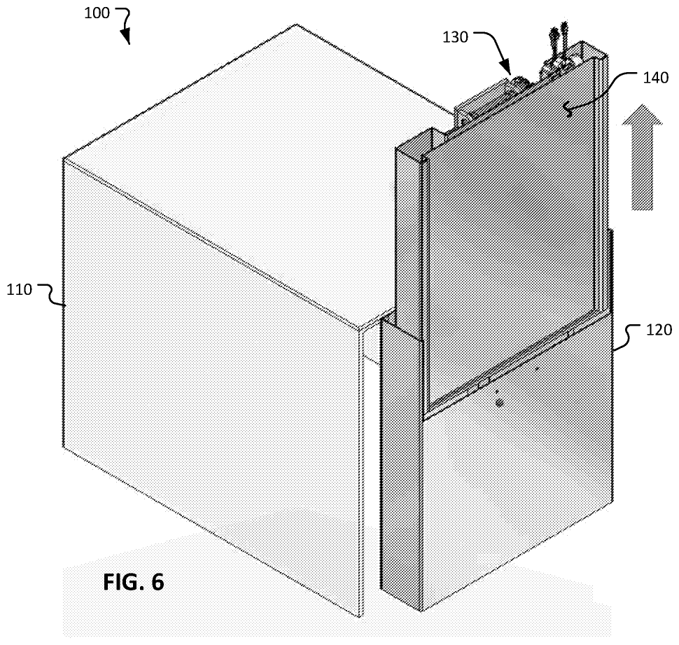

FIG. 6 shows that the casing 120 can be extended upward (telescoped) by operation of the retractable support 130.

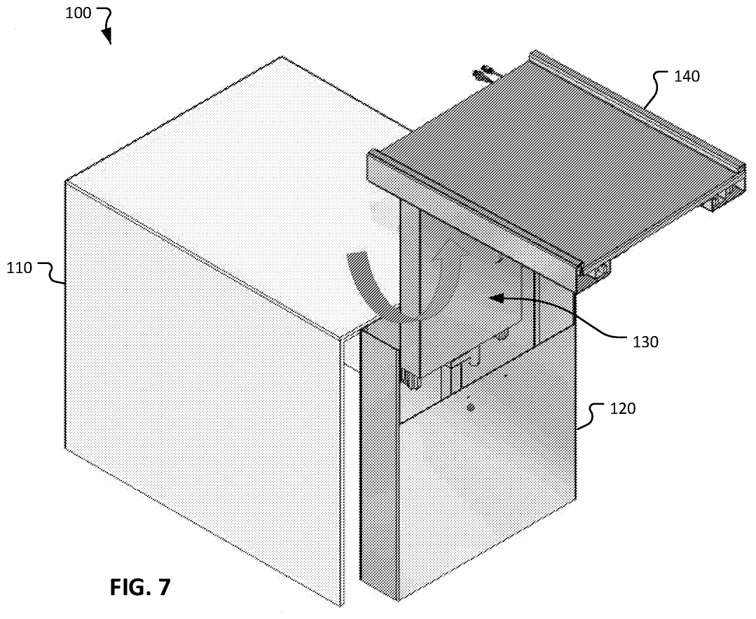

FIG. 7 shows that the middle table section 140 can be pivoted upward in relation to the retractable support 130.

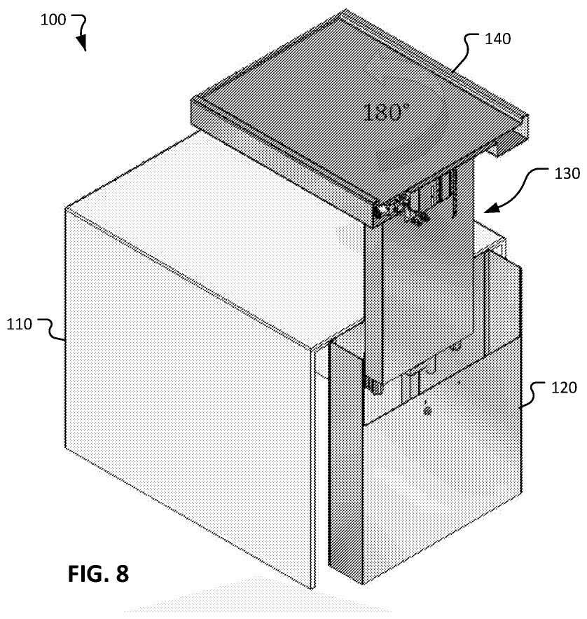

FIG. 8 shows that the middle table section 140 can be pivoted horizontally 180° relative to the retractable support 130.

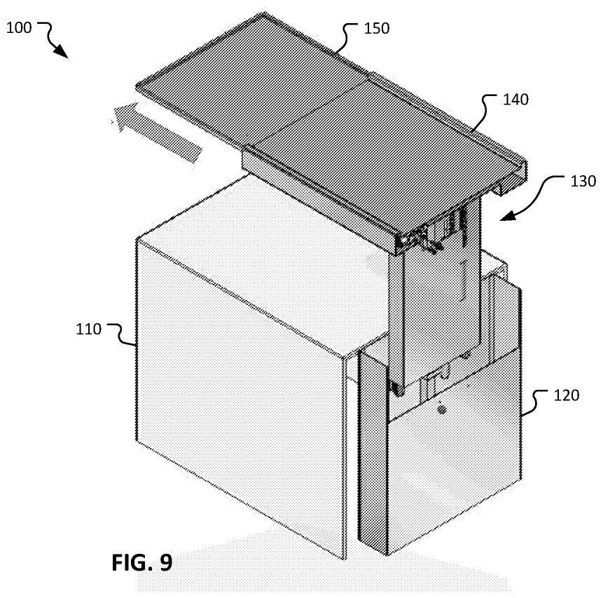

FIG. 9 shows that the front table section 150 can be horizontally extended in relation to the middle table section 140. This shows the operable configuration of the ESIT 100.

FIG. 10 shows the horizontal retraction of the front table section 150 to be adjacent to the middle table section 140.

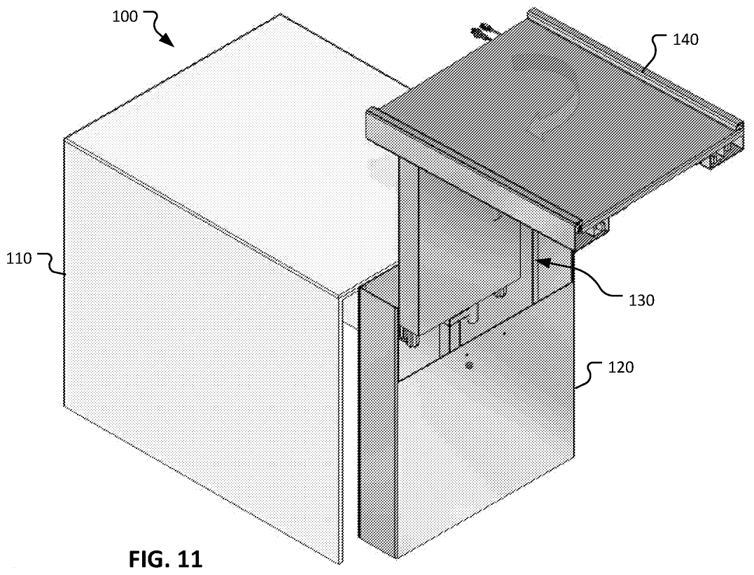

FIG. 11 shows that the middle table section 140 has been rotated horizontally 180° relative to the retractable support 130.

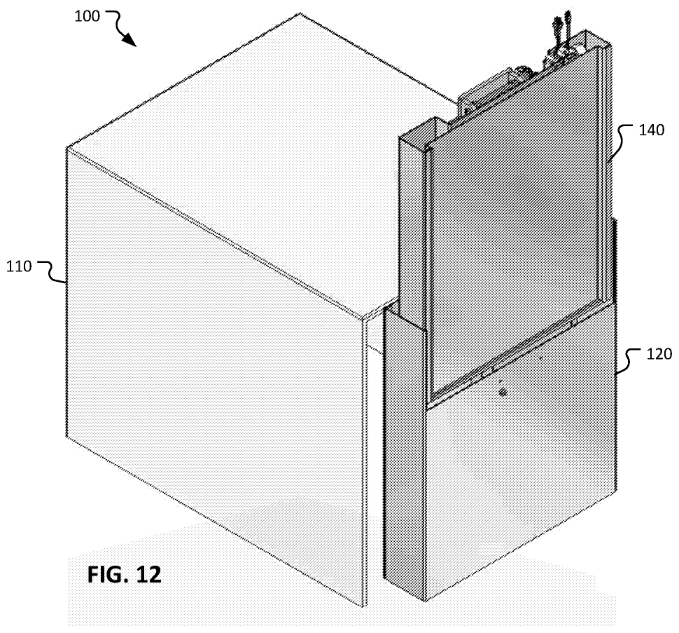

FIG. 12 shows that the middle table section 140 has been pivoted downward 90° to be in alignment with the casing 120.

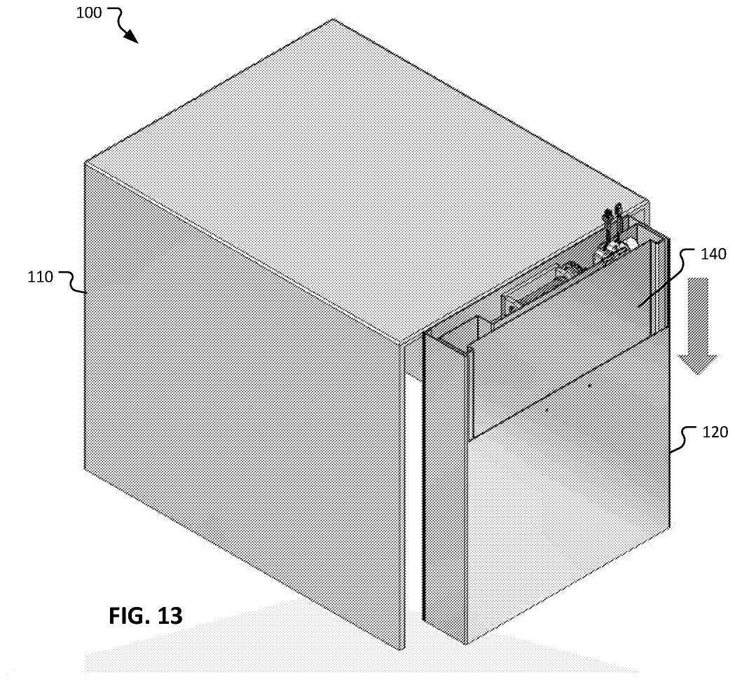

FIG. 13 shows that the middle table section 140 has been translated downward into the casing 120. After that, the casing 120 is pivoted upward 90° to positioned adjacent to the underside of the top of the base 110.

### Additional Embodiments and Optional Features

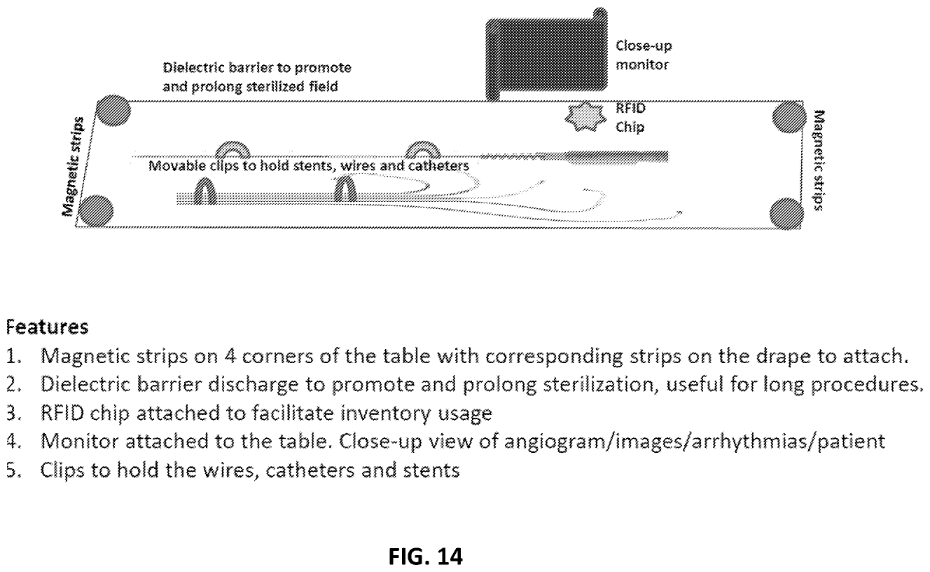

FIG. 14 depicts some additional accessories that can be used with the ESIT 100 described above. The additional accessories can also be referred to as consumables, and include the following.

The first accessory is a surgical drape that has at least two unique features. It will have four magnetic corner strips that align with corresponding strips on the ESIT 100 and will contract and expand as per the needs of the procedure or the operator. Second, there is a dielectric barrier discharge that will promote sterilization of the operating field. This is especially relevant in long procedures where the sterility of the operating field can be compromised.

Second, there will be room for attachment for a close-up monitor. This multifunctional monitor will have the capacity to serve in different roles. Importantly, it will give the operator a close-up view of the angiographic images (ultrasound, echocardiogram), scans, EKGs etc., which with the present setup are difficult due to the position and distance of the monitor from the operator. This monitor can also be used to directly supervise the patient with regards to pain, discomfort, breathing etc., monitoring of which is crucial in life-threatening procedures.

Third, a surgical drape can include a radiofrequency identification (RFID) chip to automatically identify and track the tags that accompany the catheters, wires and other consumables used during a surgical or interventional procedure. This will obviate the need for separate handling of inventory by the staff, improve efficiency and minimize losses.

Fourth, movable clips can be used to facilitate holding the catheters, wires, stents and other long instruments and prevent them from falling off the table, thereby minimizing waste and loss of revenue.

In some embodiments of the ESIT 100, artificial intelligence (“AI”) will be used to augment decision-making, control, and manage inventories and learn operator needs and behaviors. Four specific uses or implementations of the AI in relation to the ESIT 100 are listed below.

1. Management and control of inventories: Typically, inventories are managed manually. These are time-consuming and inefficient tasks. AI will learn the patterns of use of consumables in the catheter lab, and enable management and restocking of those inventories in an efficient way.

2. Ergonomic efficiency: Conventional tables are controlled manually and do not cater to a particular operator. For example, if the height of the table is low, then the operator will bend to do the case. The ESIT 100, fitted with AI, will learn the individual preferences, and automatically adjust the height of the table accordingly.

3. Monitoring of vitals: Presently, the monitors are far from the operator. The monitors fitted with the ESIT 100 will have the capability of monitoring and tracking the vitals of the patients and alert the operator if something out-of-the-ordinary is detected.

4. Adjustment of radiation shield: Based on the location of the vascular access, in procedures that require radiation the shield that blocks the radiation exposure to the operator, needs to be moved and adjusted manually. With ESIT 100 fitted with AI, this shield can be adjusted automatically based on the operator's position and location of vascular access.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

1. A surgical instrument table comprising:
a base;
a casing rotatably coupled to the base;
a first table section;
a support mechanism coupling the first table section to the casing; and
a second table section slidably coupled to the first table section.

2. The surgical instrument table of claim 1, wherein the first and second table sections are slidable into and out of an interior space of the casing.

3. The surgical instrument table of claim 1, wherein the casing is positionable adjacent to an underside surface of the base.

4. The surgical instrument table of claim 1, wherein the first and second table sections are pivotable in relation to the casing.

5. The surgical instrument table of claim 1, wherein the first and second table sections are rotatable 180° relative to the base.

6. The surgical instrument table of claim 1, wherein the movements of the casing and the first and second table sections are motorized.

7. The surgical instrument table of claim 1, wherein the movements of the casing and the first and second table sections are manually actuated.

8. A system comprising:
a surgical instrument table comprising:
a base;
a casing rotatably coupled to the base;
a first table section;
a support mechanism coupling the first table section to the casing; and
a second table section slidably coupled to the first table section; and
a surgical drape positioned on the surgical instrument table; and
a close-up monitor coupled to the surgical instrument table.

9. The system of claim 8, further comprising:
a first set of magnetic strips coupled to the surgical instrument table; and
a second set of magnetic strips coupled to the surgical drape positioned on the surgical instrument table, wherein the first set of magnetic strips correspond to the second set of magnetic strips.

10. The system of claim 8, wherein the close-up monitor is configured to provide an operator a close-up view of surgical images.
